(19) United States
(12) Patent Application Publication
(10) Pub. No.: US 2019/0365317 A1
(43) Pub. Date: Dec. 5, 2019

(54) PHYSIOLOGICAL SENSING AND THERAPEUTIC ADMINISTRATION SYSTEM AND METHOD

(71) Applicant: Shire Human Genetic Therapies, Inc., Lexington, MA (US)

(72) Inventors: Gerald Spotts, Encino, CA (US); Roman Pichler, Lexington, MA (US); Michael Nelson, Lexington, MA (US)

(21) Appl. No.: 16/480,167

(22) PCT Filed: Jan. 29, 2018
(86) PCT No.: PCT/US2018/015677

§ 371 Date: Jul. 23, 2019

Related U.S. Application Data
(60) Provisional application No. 62/453,112, filed on Feb. 1, 2017.

(51) Int. Cl. A61B 5/00; G16H 20/10

(52) U.S. Cl. CPC A61B 5/4839; G16H 40/63; A61B 5/6824; G16H 20/10

(57) ABSTRACT

Embodiments of the present disclosure relate to a physiological sensing and therapeutic administration system. The system comprises a sensor and a pharmacokinetic (PK) server. The sensor (e.g., a physiological sensor) is configured to acquire real-time physiological measurements of a patient. The PK server is configured to determine a recommended administration of a therapeutic for the patient based on the acquired real-time physiological measurements of the patient. Due to the patient metabolizing therapeutic, the therapeutic has a time-varying concentration level in the patient. For instance, the concentration of the therapeutic in the patient decreases after an infusion.

### RELATED APPLICATION

This application claims the benefit of U.S. Provisional Application No. 62/453,112, filed on Feb. 1, 2017. The entire teachings of the above application are incorporated herein by reference.

### BACKGROUND

In recent years, health and fitness has grown tremendously. The growth has occurred, in large part, due to a better understanding of the benefits of good fitness to overall health and wellness. For example, according to recent expert recommendations, adults should perform 10,000 steps per day. Unfortunately, fitness activities can be dangerous for a percentage of the population. For instance, some members of the population suffer from a disease or genetic disorder that requires consistent monitoring and maintenance of a therapeutic plasma concentration level. Accordingly, such persons may experience an adverse event because a fitness activity (e.g., exercising and/or playing a sport) can unpredictably metabolize therapeutic plasma to below desired levels, thus jeopardizing their health and safety.

Notably, patients with hemophilia A have a genetic deficiency that causes low levels of clotting factor VIII. Clotting factor VIII is a blood-clotting protein that is activated in response to an injury or bleed. Individuals with relatively low levels of clotting factor VIII are susceptible to internal or external episodes of prolonged bleeding resulting from an injury and/or spontaneous bleeding without a cause. Accordingly, participation in a fitness activity can result in internal bleeding of joints, muscles, and organs and cause permanent damage, disfigurement, or even death.

Treatment of patients with hemophilia A (or patients that otherwise have low levels of clotting factor VIII) includes providing these patients with periodic infusions of a clotting factor concentrate (e.g., therapeutic plasma protein). The clotting factor concentrate acts as a replacement or supplement for the patient's natural occurring clotting factor VIII. One example of such a therapeutic plasma protein is Shire's ADVATE drug. In some instances, patients receive the therapeutic plasma protein in response to having an uncontrolled internal bleed. Alternatively, patients may be prescribed a prophylactic treatment regimen of the therapeutic plasma protein to reduce the possibility of future bleeds. However, prophylactic treatment regimens are based on historical biological, demographic, activity information of a patient. Thus, such treatment regimens cannot account for spontaneous participation in an activity or lifestyle changes because such changes cannot be modeled into the creation of the treatment regime.

Currently, patients are provided with electronic diaries to record infusion events and bleed events. Unfortunately, such diaries do not provide actionable data from which a patient is able to plan activity levels. For example, with current systems, patients are unable to ascertain a risk of bleeding if the patient were to participate in an activity such as playing soccer at any point in time after an infusion event. Furthermore, even if a patient does participate in an activity without experience an adverse event, the patient is unable to ascertain how their therapeutic plasma concentration level has changed due to the participation.

Thus, with current electronic diaries, patients cannot determine their factor level at any given point in time after a prophylactic infusion event, significantly limiting the usefulness of the information provided by such sources. Moreover, patients are not able to make an informed adjustment of an administration of the clotting factor (e.g., by taking an on-demand infusion to prevent a bleed or adjusting a prophylactic infusion schedule).

### SUMMARY

Embodiments of the present disclosure provide a physiological sensing and therapeutic administration system that provides a customized recommendation for an administration of a therapeutic for a patient by tracking physiological data such as physical activity in real-time. Notably, the system determines a recommended administration based on a patient's real-time activity levels. As such, embodiments of the invention are able to monitor a patient's activity levels in real-time thus providing a much more reliable method of providing customized recommendations than conventional methods. Advantageously, the disclosed system allows a patient to live an active lifestyle without suffering from an adverse event due to a low therapeutic plasma concentration level. In a case which the patient suffers from hemophilia A, the system ensures that the patient maintains a healthy plasma concentration level of clotting factor VIII.

For example, a patient may participate in a physically demanding activity (e.g., a sport such as soccer) that causes the clotting factor VIII to be metabolized at a rate quicker than, for example, a sedentary activity (e.g., reading a book). Accordingly, the system, using physiological measurements, can determine or predict a time-varying concentration level of the clotting factor VIII based on the patient's activity level. Based on the determined or predicted concentration level, the system recommends an infusion of clotting factor VIII.

The system can further determine a frequency of the activity and compare the frequency with historical activity measurements. Based on the comparison, the system can determine whether the recommended administration should be an on-demand infusion (e.g., if the activity is random and infrequent) or an adjusted prophylactic infusion schedule (e.g., if the activity is frequent and a change from the patient's historical activity levels).

Accordingly, the physiological sensing and therapeutic administration system enables patients suffering from an illness, disease, or genetic disorder to live healthy and active lifestyles by using real-time physiological monitoring tools. Particularly, the real-time physiological monitoring tools obtain live physiological data feeds of these types of patients. The live physiological data feeds enable the system to provide the patients with “in the moment” customized, accurate, and reliable recommendations for a therapeutic administration. By providing such patients with therapeutic administration recommendations during their daily activities, the system removes a need for such patients to plan their daily activities. Thus, the real-time physiological monitoring and therapeutic recommendations provided by embodiments of the invention allow such patients to dynamically adjust activity levels (whether it is spontaneous or a part of a change in lifestyle) without suffering from an adverse event due to a low therapeutic plasma concentration level. Moreover, the system enables such patients to live worry free lifestyles similar to those that do not suffer from a disease or genetic disorder.

In one embodiment, a physiological sensing and therapeutic administration system comprises a sensor and a pharmacokinetic (PK) server. The sensor is configured to acquire real-time physiological measurements of a patient. For example, the system can leverage the vast ecosystem of consumer wearable technologies (e.g., the Fitbit®) to acquire real-time physiological measurements (e.g., steps). The PK server then determines a recommended administration of a therapeutic for the patient based on the acquired real-time physiological measurements of the patient. Because the therapeutic has a time-varying concentration level in the patient, the system processes the “steps” information to quantify an amount and intensity of the patient's physical activity to determine a current rate at which the patient is metabolizing the therapeutic.

As such, in an aspect, the system can further comprise a therapeutic monitoring tool configured to determine a time-varying therapeutic concentration level in the patient at any given point in time based on a PK profile of the patient and/or the acquired real-time physiological measurements of the patient.

In an example, the recommended administration of the therapeutic drug can include a timing and/or quantity component. The timing and/or quantity component can be a function of the acquired real-time physiological measurements. In one aspect, the timing component is a customized prophylactic dosing regimen of the therapeutic for the patient. In a further aspect, the timing component is an on-demand dosing of the therapeutic based on the time-varying therapeutic concentration level in the patient. In other aspects, the quantity component can be an amount of the therapeutic determined based on a function of the timing component and/or the acquired real-time physiological measurements.

In some aspects, the PK server can be further configured to quantify an amount and intensity of the patient's physical activity based on the physiological measurements. In additional aspects, the PK server can be further configured to determine the recommended administration of the therapeutic for the patient based on the amount and intensity of the patient's physical activity. In further aspects, the therapeutic dosing tool can be configured to determine the time-varying therapeutic concentration level in the patient based on the amount and intensity of the patient's physical activity.

In additional aspects, the sensor can be coupled to the patient's body at a location based on a type of the physiological measurements. For example, a fitness wearable can be worn on a patient's wrist. Also, the sensor can include circuitry and/or hardware that is specific to enable real-time acquisition of the type of the physiological measurements.

Another embodiment of the present disclosure includes a physiological sensing and therapeutic administration method. The method comprises acquiring, via a physiological sensor, real-time physiological measurements of a patient. The method also comprises determining, via one or more processors, a recommended administration of a therapeutic for the patient based on the acquired real-time physiological measurements of the patient. The therapeutic has a time-varying concentration level in the patient.

The method of claim can further comprise determining, via the one or more processors, a time-varying therapeutic concentration level in the patient at any given point in time based on a PK profile of the patient and/or the acquired real-time physiological measurements of the patient.

In one aspect, the recommended administration of the therapeutic can include a timing and/or quantity component. For example, the timing and/or quantity component can be a function of the acquired real-time physiological measurements. The timing component can be a customized prophylactic dosing regimen of the therapeutic for the patient. In other aspects, the timing component can be an on-demand dosing of the therapeutic based on the time-varying therapeutic concentration level in the patient. In alterative aspects, the quantity component can be an amount of the therapeutic determined based on a function of the timing component and/or the acquired real-time physiological measurements.

In some aspects, the method can further comprise quantifying, via the one or more processors, an amount and intensity of the patient's physical activity based on the physiological measurements. In additional aspects, the method can further comprise determining the recommended administration of the therapeutic for the patient based on the amount and intensity of the patient's physical activity. In further aspects, the method can comprise determining, via the one or more processors, the time-varying therapeutic concentration level in the patient based on the amount and intensity of the patient's physical activity.

The method can also comprise coupling the physiological sensor to the patient's body at a location based on a type of the physiological measurements. In an aspect, the physiological sensor can include circuitry and/or hardware that are specific to enable real-time acquisition of the type of the physiological measurements.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 illustrates an example environment in which a physiological sensing and therapeutic system operates, according to an example embodiment of the present disclosure.

FIG. 2 is a logical block diagram of a biometric monitoring device according to an example embodiment of the present disclosure.

FIG. 3 illustrates an example sensor for use with a biometric monitoring device according to an example embodiment of the present disclosure.

FIG. 4 is a flow diagram of a method for physiological sensing and therapeutic administration according to an example embodiment of the present disclosure.

FIG. 5 is a flow diagram of a method for determining a recommended administration of a therapeutic based on an amount and intensity of a patient's physical activity according to an example embodiment of the present disclosure.

FIG. 6 is a detailed block diagram of an example remote server, therapeutic monitoring tool, and/or ecosystem monitoring system in accordance with an example embodiment of the present disclosure.

### DETAILED DESCRIPTION

A description of example embodiments of the present disclosure follows.

The present disclosure relates a physiological sensing and therapeutic administration system. The system determines and recommends an administration of a therapeutic based on real-time physiological measurements collected by physiological sensors. The sensors can range from widespread consumer wearable electronics (e.g., the Apple Watch®) to advanced medical sensors (e.g., the pocket ECG™). Armed with such a recommendation system, patients are better equipped, for example, to participate in a physically demanding activity (e.g., a sport such as soccer).

For example, for patients suffering from hemophilia A, the system recommends an administration of clotting factor VIII based on the patients' activity levels determined from the real-time physiological measurements. Particularly, the system can receive “steps” information (e.g., a number of steps and frequency of the steps with respect to each other) and quantify the patient's activity levels while the patient is participating in the activity. This real-time quantification allows the system to accurately determine the patient's clotting factor VIII plasma concentration. For instance, the system can use the quantified activity levels to determine the patient's metabolism of the clotting factor VIII based on the patient's a pharmacokinetic (PK) profile.

Accordingly, the disclosed system enables such patients to live active lifestyles by recommending, for example, “in the moment” administration of clotting factor VIII based on real-time physiological measurements. Advantageously, the system ensures that those patients maintain a healthy level of clotting factor VIII such that they can live lifestyles that are not inhibited due to suffering from hemophilia A.

Although embodiments of the present disclosure is described herein with respect to a patient suffering from hemophilia A, a skilled artisan understands that the embodiments can be applied to a patient suffering from any disease or genetic disorder requiring administration of a therapeutic (e.g., a diabetic patient that requires regular infusions of insulin).

As used herein, the term “clotting factor VIII”, “FVIII”, or “rAHF” refers to any FVIII molecule that has at least a portion of the B domain intact, and which exhibits biological activity that is associated with native FVIII. In one embodiment of the disclosure, the FVIII molecule is full-length FVIII. The FVIII molecule is a protein that is encoded by DNA sequences capable of hybridizing to DNA encoding FVIII:C. Such a protein may contain amino acid deletions at various sites between or within the domains A1-A2-B-A3-C1-C2. The FVIII molecule may also be an analog of native clotting factor FVIII, wherein one or more amino acid residues have been replaced by site-directed mutagenesis.

The term “recombinant Factor VIII” (rFVIII) may include any rFVIII, heterologous or naturally occurring, obtained via recombinant DNA technology, or a biologically active derivative thereof. As used herein, “endogenous FVIII” includes FVIII which originates from a mammal intended to receive treatment. The term also includes FVIII transcribed from a transgene or any other foreign DNA present in the mammal. As used herein, “exogenous FVIII” or therapeutic plasma protein includes clotting factor FVIII that does not originate from a mammal.

The FVIII molecule exists naturally and in therapeutic preparations as a heterogeneous distribution of polypeptides arising from a single gene product. The term “clotting factor VIII” as used herein refers to all such polypeptides, whether derived from blood plasma or produced through the use of recombinant DNA techniques and includes, but is not limited to FVIII mimetics, fc-FVIII conjugates, FVIII chemically modified with water soluble polymers, and other forms or derivatives of FVIII. Commercially available examples of therapeutic preparations containing FVIII include those sold under the trade names of ADVATE, HEMOFIL M, and RECOMBINATE (available from Shire, Bannockburn, Ill., U.S.A.). Other preparations comprise primarily a single subpopulation of FVIII molecules, which lack the B domain portion of the molecule.

The FVIII molecules useful for the present disclosure include a full-length protein, precursors of the protein, biologically active or functional subunits or fragments of the protein, and/or functional derivatives thereof, as well as variants thereof as described herein below. Reference to clotting factor FVIII is meant to include all potential forms of such proteins and wherein each of the forms of FVIII has at least a portion or all of the native B domain sequence intact.

“Dosing interval,” as used herein, means an amount of time that elapses between multiple doses being administered to a patient. The dosing interval for administering a therapeutic plasma protein including clotting factor VIII may be at least about every one, two, three, four, five, six, seven, eight, nine, ten, eleven, twelve, thirteen, or fourteen days or longer. The dosing interval may change based on changing conditions/characteristics of a patient, changes to a minimally acceptable (e.g., target trough) concentration of the therapeutic plasma protein within a patient, and/or changes to a dosage.

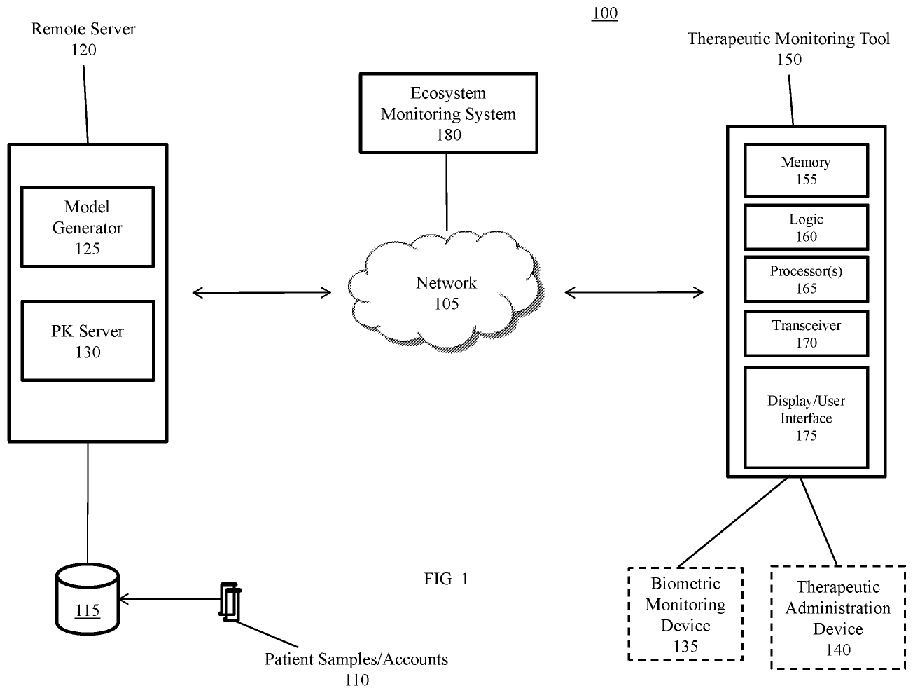

FIG. 1 illustrates a physiological sensing and therapeutic system 100 according to an example embodiment of the present disclosure. The system 100 determines and recommends an administration of a therapeutic (e.g., clotting factor VIII) based on real-time physiological measurements collected from a biometric monitoring device 135. To that end, the system 100 comprises a remote server 120, ecosystem monitoring system 180, therapeutic monitoring tool 150, biometric monitoring device 135, and therapeutic administration device 140.

The remote server 120 includes a model generator 125 and a PK server 130. The remote server 120 is communicatively coupled to a data store 115 that stores, for example, patent medical samples and patient accounts 110. The model generator 125 is configured to generate one or more patient pharmacokinetic (PK) models based upon sampled patient data 110. The PK server 130 is configured to provide patients, healthcare providers, and/or sales representatives with a therapeutic monitoring tool 150 based upon the one or more pharmacokinetic models. In the illustrated embodiment, the PK server 130 transmits the patient PK model to the therapeutic monitoring tool 150 via a network 105 (e.g., the Internet). In other embodiments, the PK server 130 hosts the PK profile, which is accessible by the therapeutic monitoring tool 150. In these other embodiments, the PK server 130 may include a single server, or alternatively, may be distributed within a cloud computing framework.

The example PK server 130 and/or the model generator 125 may be communicatively coupled to a database 115 configured to store the patient pharmacokinetic (PK) models. The database 115 may include any type of computer-readable medium, including RAM, ROM, flash memory, magnetic or optical disks, optical memory, or other storage medium. The example database 115 may also store information generated in response to users using the tool 150 including, for example, patient information, dosing regimes, etc. In some instances, the database 115 may be managed by a separate third-party storage provider.

In some instances, the PK server 130 and/or the model generator 125 may be provided by the same server (e.g., the remote server 120) and/or processor and/or operated by the same entity. In these instances, the functionality of the model generator 125 may operate in conjunction with the functionality of the PK server 130. For instance, the model generator 125 may periodically update pharmacokinetic models with therapeutic plasma protein dosing information and/or patient information received in the PK server 130 via the tool 150.

In some example embodiments, a pharmacokinetic (PK) model is used to approximate pharmacokinetic (PK) profiles of patients. For instance, current methods to determine a patient-specific pharmacokinetic profile for hemophilia A include performing multiple blood tests. These blood tests include performing an initial blood draw to determine a clotting factor VIII baseline in a patient. Then, after therapeutic plasma protein is administered, five or more blood draws are performed over a 48-hour post-infusion period. As can be appreciated, such a procedure is especially taxing on a patient, healthcare provider, and lab because of the numerous separate blood draws. Accordingly, the example model generator 125 is configured to generate relatively accurate pharmacokinetic models based upon a sample of patients with varying ages, body weights, genders, and activity levels. These models are then used to determine or approximate a pharmacokinetic profile of a patient without having to subject a patient to all of the blood draws and subsequent analysis.

In an embodiment, the PK models are determined using patient samples 110 selected from one or more sets of patient data. The patient samples 110 may be, for example, selected among patients who have already been subscribed a therapeutic dosing regimen using the above described blood draw procedure. The patient samples 110 may also include patients specifically selected to go through the blood draw procedure for the purpose of creating the models. The patient samples 110 may include patients from one hospital or medical system and/or patients associated from multiple hospitals, medical systems, geographic regions, etc.

The patient samples 110 include data for patients of varying ages, body weights (or body mass index (“BMI”), medical conditions, clinical laboratory data, genders, and/or activity levels. In the example described herein, sample patient ages vary between 2 and 100 years of age. In some embodiments, the data for the patients may be separated into children and adult age brackets such that a separate model is generated for each bracket. The patient data may additionally or alternatively be partitioned based on weight, gender, and/or activity level.

As mentioned, the example patient samples 110 include a determination of clotting factor VIII before therapeutic plasma protein is infused into the patients. Then, post infusion blood samples are collected from each patient after certain durations of time. It should be appreciated that in other examples, the blood samples may be collected at different times and/or the number of blood samples collected may be fewer or greater. For instance, fewer blood samples may be collected from children.

The example model generator 125 creates a PK patient model by performing a Bayesian analysis that uses previous knowledge of clotting factor VIII in the sampled patients over time after an infusion of the therapeutic plasma protein. In some instances, the model generator 125 is configured to analyze each patient's sampled dosing history in conjunction with pre-infusion clotting factor VIII levels, so that washout data is not needed to construct the PK models. In other embodiments, the model generator 125 may use patient washout data in conjunction with the post-infusion clotting factor VIII levels to create one or more pharmacokinetic models. Patient washout data corresponds to a baseline where the patient does not include the therapeutic plasma protein in their system.

The example model generator 125 creates the one or more PK models using, for example, the patient sample data. The model generator 125 may combine the individual patient samples 110 into one or more population profiles (e.g., age sets, weight sets, activity level sets, endogenous clotting factor VIII level, etc.), which is then used as a basis for the respective pharmacokinetic model. For instance, the model generator 125 may group the patient samples 110 for different ages, weights, and/or activity levels into different sets. The model generator 125 then performs covariate and statistical modeling on the grouped patient samples 110 of each set to create a population pharmacokinetic model for that set, as described in a white paper titled “Population pharmacokinetics of recombinant factor VIII—the relationships of pharmacokinetics to age and body weight”, by Bjorkman et al., the entirety of which is incorporated herein by reference. It should be appreciated however, that the model generator 125 may model the sampled data 110 using other Bayesian analysis techniques (e.g., a naïve Bayes classifier).

In the illustrated example, the covariate model used by the model generator 125 determines relationships between pharmacokinetic parameters (e.g., how quickly therapeutic plasma protein is metabolized, endogenous clotting factor VIII level, etc.) and patient characteristics (e.g., age, body weight, clinical laboratory data, gender, activity level, etc.). The model generator 125 uses a statistical model to determine variance in pharmacokinetic parameters among the sampled patients in addition to residual variance as a result of biological variability between patients, measurement errors, and errors within the fit of the sampled data 110 to the pharmacokinetic model.

The example model generator 125 is configured to perform the covariate and statistical modeling using non-linear mixed effects modeling with a first-order integral approximation method, as provided in SAS® software (NLMIXED procedure). In the illustrated example, the model generator 125 uses a two-compartment model. In other examples, the model generator 125 may use a single compartment model or three or more compartment models. In the illustrated two-compartment example, the first compartment includes pharmacokinetic parameters of clearance (“CL”) and volume of distribution (V1). CL refers to the amount of time for a patient to metabolize the therapeutic plasma protein in milliliters (“mL”) per hour per kilogram (“kg”). In other words, clearance is a measure of efficiency and rate at which a therapeutic plasma protein is removed or eliminated from a patient.

Responsive to creating one or more pharmacokinetic models, the model generator 125 provides the pharmacokinetic model(s) to the PK server 130. The transmission may be over a private network, such as a local area network, or over a public network, such as an Internet. The model generator 125 may also store the models to the database 115, which is also accessible by the PK server 130 via one or more interfaces. In other instances, the model generator 125 may be integrated with the PK server 130.

The example model generator 125 may refine the models for each patient. For instance, the PK server 130 may receive patient specific information including, weight, age, gender, endogenous clotting factor VIII level, and dosing level for previous treatments. The model generator 125 uses the previous treatment information (e.g., dosing amounts, intervals, etc.) to refine or adjust the model such that dosing recommendations and a pharmacokinetic profile are more aligned to the specific patient but still account for potential patient variance. The model generator 125 transmits the patient-specific model to the PK server 130.

Alternatively, the PK server 130 may be configured to create patient-specific models using the pharmacokinetic model provided by the model generator 125 to account for the patient-specific pharmacokinetic variance. In this manner, one or more base models are refined or adjusted by the PK server 130 responsive to receiving previous treatment information for a specific patient. The PK server 130 may be configured to store the patient-specific model to the database 115 for subsequent uses by the same healthcare provider or other healthcare providers.

Once a PK profile for a patient is generated, the PK server is configured to transmit the PK profile to the therapeutic monitoring tool 150. In some embodiments, the PK server 130 can encrypt the data file prior to transmission. The encryption can be specific to a particular patient such that the therapeutic monitoring tool 150 can only open and process a received PK profile if the tool 150 has a patient specific authentication key. Once the tool 150 is activated, the tool 150, via processor(s) 165, generates an interactive user interface 175 for display on the tool 150.

The ecosystem monitoring system 180 is coupled to the network 105 and in communication with both the remote server 120 and therapeutic monitoring tool 150. The system 180 can provide notification to a pharmacist to prepare the particular clotting factor VIII drug for purchase by a patient. For example, the system 180 can determine that the patient has a threshold amount of the drug left such that the patient will be in need of the drug in the near future. Similarly, the system 180 can contact a physician to ensure that physician has real-time information associated with the patient. Accordingly, the physician can take immediate actions to care of the patient if a need arises.

The therapeutic monitoring tool 150 can be communicatively coupled to an infusion pump 140 and a biometric monitoring device 135. The infusion pump 140 can be configured to automatically administer a particular clotting factor VIII drug based on a dosing regimen/treatment schedule. In some embodiments, the infusion pump 140 can be configured to administer a dose of the particular clotting factor VIII drug in response to results of a biological sample collected by the biometric monitoring device 135. For example, the biometric device 135 can collect a blood sample and determine a factor VIII level of the patients. In response to the amount, the infusion pump 140 can administer a dose of the particular clotting factor VIII drug.

The physiological sensing and therapeutic administration system 100 can be further configured to recommend and/or control an administration of clotting factor VIII based on the patients' activity levels determined from the real-time physiological measurements collected by the biometric monitoring device 135.

For example, a patient may participate in a physically demanding activity (e.g., a sport such as soccer) that causes the clotting factor VIII to be metabolized at a rate quicker than, for example, a sedentary activity (e.g., reading a book). Accordingly, the system 100, using physiological measurements collected from the biometric monitoring device 135, can determine or predict a time-varying concentration level of the clotting factor VIII based on the patient's activity level. Using the determined or predicted concentration level, the system 100 recommends an infusion of clotting factor VIII. In particular, the system 100 can provide a notification or alert to the patient via the user interface 175. The system 100 can also administer the clotting factor VIII via the administration device 140.

The biometric monitoring device 135 can be contained in a housing (not shown), which may be worn or held by a user. The housing may be in the form of a wristband, a clip on device, a wearable device, or may be held by the user either in the user's hand or in a pocket or attached to the user's body. The biometric monitoring device 135 includes device components, which may be in the form of logic, storage, and glue logic, one or more processors, microelectronics, and interfacing circuitry. The components may be specific to the type of the physiological measurements being collected to recommend and/or control an administration of a therapeutic (e.g., clotting factor VIII). Accordingly, the biometric device 135 can include one or more sensors.

The sensors may be in the form of motion detecting sensors. In some embodiments, a motion sensor can be one or more of an accelerometer, or a gyroscope, or a rotary encoder, or a calorie measurement sensor, or a heat measurement sensor, or a moisture measurement sensor, or a displacement sensor, or an ultrasonic sensor, or a pedometer, or an altimeter, or a linear motion sensor, or an angular motion sensor, or a multi-axis motion sensor, or a combination thereof. The sensors can also be in the form of biometric sensors to measure physiological characteristics of the patient that is using the biometric monitoring device 135.

As mentioned, the sensors can detect motion of the biometric monitoring device 135. The motion can be activity of the user, such as walking, running, stair climbing, etc. Accordingly, the logic may include activity tracking logic. The activity tracking logic can include logic that is configured to process motion data produced by sensors, so as to quantify the motion and produce identifiable metrics associated with the motion. Some motions will produce and quantify various types of metrics, such as step count, stairs climbed, distance traveled, very active minutes, calories burned, etc.

The biometric monitoring device 135 can communicate with the therapeutic monitoring tool 150 and/or server 120 using the wireless transceiver (not shown). The wireless transceiver allows the biometric monitoring device 135 to communicate using a wireless connection, which is enabled by wireless communication logic. The wireless communication logic can be in the form of a circuit having radio communication capabilities. The radio communication capabilities can be in the form of a Wi-Fi connection, a Bluetooth connection, a low-energy Bluetooth connection, or any other form of wireless tethering or near field communication. In still other embodiments, the biometric monitoring device 135 can communicate with other computing devices using a wired connection (not shown).

The therapeutic monitoring tool 150 is a computing device that is capable of communicating wirelessly with biometric monitoring device 135 and with the network 105 (e.g., the Internet). The therapeutic monitoring tool 150 can support installation and execution of applications (e.g., APPs, mobile APPs, etc.). Such applications can include a therapeutic monitoring application. The application can be downloaded from the remote server 120. The server 120 can be a specialized server or a server that provides applications to devices, such as an application store. Once the application is installed in the therapeutic monitoring tool 150, the therapeutic monitoring tool 150 can communicate or be set to communicate with biometric monitoring device 135. The therapeutic monitoring tool 150 can be a smartphone, a handheld computer, a tablet computer, a laptop computer, a desktop computer, or any other computing device capable of wirelessly interfacing with biometric device 135.

In one embodiment, the therapeutic monitoring tool 150 communicates with the biometric monitoring device 135 over a Bluetooth connection. In one embodiment, the Bluetooth connection is a low energy Bluetooth connection (e.g., Bluetooth LE, BLE, or Bluetooth Smart). Low energy Bluetooth is configured for providing low power consumption relative to standard Bluetooth circuitry. Low energy Bluetooth uses, in one embodiment, a 2.4 GHz radio frequency, which allows for dual mode devices to share a single radio antenna. In one embodiment, low energy Bluetooth connections can function at distances up to 50 meters, with over the air data rates ranging between 1-3 megabits (Mb) per second. In one embodiment, a proximity distance for communication can be defined by the particular wireless link, and is not tied to any specific standard. It should be understood that the proximity distance limitation will change in accordance with changes to existing standards and in view of future standards and/or circuitry and capabilities.

The therapeutic monitoring tool 150 can also communicate with the remote server 120 using an Internet connection. The Internet connection of the therapeutic monitoring tool 150 can include cellular connections, wireless connections such as Wi-Fi, and combinations thereof (such as connections to switches between different types of connection links).

As stated above, the remote server 120 is also provided, which is interfaced with the network 105 (e.g., the Internet). The remote server 120 can include a number of applications that service the biometric monitoring device 135, and the associated users of the biometric monitoring device 135 by way of user accounts. For example, the remote server 120 includes the storage 115 that includes various user profiles associated with the various user accounts 110.

The information can include, without limitation, device-user account pairing, system configurations, user configurations, settings and data, etc. The storage 115 will include any number of user profiles, depending on the number of registered users having user accounts for their respective therapeutic monitoring tool 150. It should also be noted that a single user account can have various or multiple devices associated therewith, and the multiple devices can be individually customized, managed and accessed by a user.

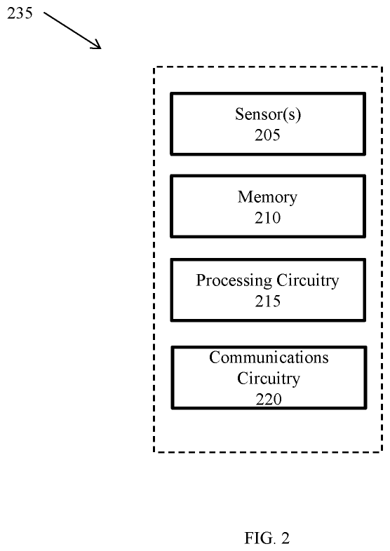

FIG. 2 is a logical block diagram of a biometric monitoring device 235 according to an example embodiment of the present disclosure. The biometric monitoring device includes one or more sensors 205, memory 210, processing circuitry 215, and communications circuitry 220.

The biometric monitoring device 235 is configured to measure, calculate, assess and/or determine physiologic data using data acquired from the one or more sensors 205. In particular, the sensors 205 are configured to sense, measure and/or detect physiologic data, for example, data which is representative of body fat, fat-free mass, hydration, body cell mass, height, eye color, heart rate, respiratory rate, blood pressure, arterial stiffness, and/or therapeutic plasma concentration levels. In addition thereto, or in lieu thereof, biometric monitoring device 235 of the present invention may detect measure and/or sense (via appropriate sensors) other physiologic data; all such physiologic data or parameters, whether now known or later developed, and fall within the scope of the present invention.

The biometric monitoring device 235 may be programmed or configured (for example, by the user via user interface 175 of the therapeutic monitoring device 150 to enable or engage (or disable or disengage) one or more physiological sensors 205 and/or enable or disable the monitoring, calculating and/or determining of one or more physiological parameters (based on or using data from such sensors 205).

For example, where biometric monitoring device 235 includes a body fat sensor having electrodes for implementing BIA, it may be advantageous to disable such sensor where the user is pregnant or is equipped with a pace maker. In this regard, pregnant women are often discouraged from taking BIA measurements. However, the body fat sensor may be enabled for male patients or non-pregnant female patients.

As discussed herein, physiological sensor(s) may sense, detect, assess and/or obtain data which is representative of physiologic information of the patient (for example, weight, body fat, blood pressure, pulse rate, blood sugar and the waveform shape corresponding to the heart beat). The biometric monitoring device 235 of this embodiment may include all permutations and combinations of sensors (for example, one or more physiological sensor(s) 205).

The sensors 205 are electrically coupled to processing circuitry 215 (which may also include control circuitry to control the operations of biometric monitoring device 235). The processing circuitry 215 calculates, assesses and/or determines physiologic information using data sensed, detected and/or measured from physiological sensors 205. The processing circuitry 215 may employ any technique now known or later developed to calculate such biometric or physiologic information.

For example, where biometric monitoring device 235 includes a heart rate sensor, processing circuitry 215 may employ data from the heart rate sensor to calculate, assess and/or determine the user's heart rate using, for example, ballistocardiography. Based on the output of such sensors, processing circuitry 215 may calculate, assess and/or determine the user's heart rate and store (e.g., in the memory 210) and/or output such information (e.g., to therapeutic monitoring tool 150 via the communications circuitry 220).

The processing circuitry 215 may be discrete or integrated logic, and/or one or more state machines, processors/controllers (suitably programmed) and/or field programmable gate arrays (or combinations thereof); indeed, any circuitry (for example, discrete or integrated logic, state machine(s), processor(s)/controller(s) (suitably programmed) and/or field programmable gate array(s) (or combinations thereof)) now known or later developed may be employed to calculate, determine, assess and/or determine the physiologic information of the user based on sensor data. In addition thereto, or in lieu thereof, the processing circuitry 215 may control the physiologic sensors 205 and/or implement user commands as described herein. In operation, the processing circuitry 215 may perform or execute one or more applications, routines, programs and/or data structures that implement particular methods, techniques, tasks or operations described and illustrated herein. The functionality of the applications, routines or programs may be combined or distributed. Further, the applications, routines or programs may be implementing by the processing circuitry 215 using any programming language whether now known or later developed, including, for example, assembly, FORTRAN, C, C++, and BASIC, whether compiled or uncompiled code; all of which are intended to fall within the scope of the present invention.

The biometric monitoring device 235 uses the communication circuitry 220 (wireless and/or wired) to transmit biometric or physiologic data and/or receive, for display via the therapeutic monitoring tool 150 control signaling. The communication circuitry 220 may implement or employ any form of communications (for example, wireless, optical, or wired) and/or protocol (for example, standard or proprietary (for example, Bluetooth, ANT, WLAN, power-line networking, cell phone networks, and Internet based and/or SMS) now known or later developed, all forms of communications and protocols are intended to fall within the scope of the present invention.

In one preferred embodiment, biometric monitoring device 235 is a multi-protocol LAN to WAN gateway where local devices can be Bluetooth, ANT, ZigBee, etc. and the biometric gateway communicates to the Internet via or over a communication path (for example, a cell phone network, WLAN, etc.). The biometric monitoring device 235 may operate as an “open hotspot” so that no user setup is required. For instance, a user may have elsewhere established a network account (e.g., www.fitbit.com or another website) to an external device (e.g., a Fitbit Tracker) through its unique device ID, then the gateway automatically recognizes the device and sends data to the suitable, predetermined, associated and/or correct account and location. The data may go directly to the destination (for example, via the Internet) or through an intermediary first. Destinations or intermediaries could be other devices or a network service (e.g., www.fitbit.com).

The communication circuitry 220 may upload data and/or commands to and/or download data and/or commands from, for example, selected websites, health professionals, or health oriented monitoring groups/organizations or specialists, and/or the like (hereinafter collectively “third party” or “third parties”). In this way, biometric monitoring device 235 may manually or automatically provide physiologic data to such third parties.

The communication circuitry 220 may also facilitate programming of biometric monitoring device 235, for example, programming the device to acquire selected physiologic data (for example, via enabling and/or disabling selected physiological sensors) and/or calculate, monitor and/or determine selected physiological parameters (for example, via enabling or disabling processing circuitry 215 accordingly). The programming of biometric monitoring device 235 may be via the patient or third party. In this way, for example, a third party may customize or tailor the acquisition of physiologic data based on the patient, the situation (for example, physical condition of the patient), and the acquisition of desired information.

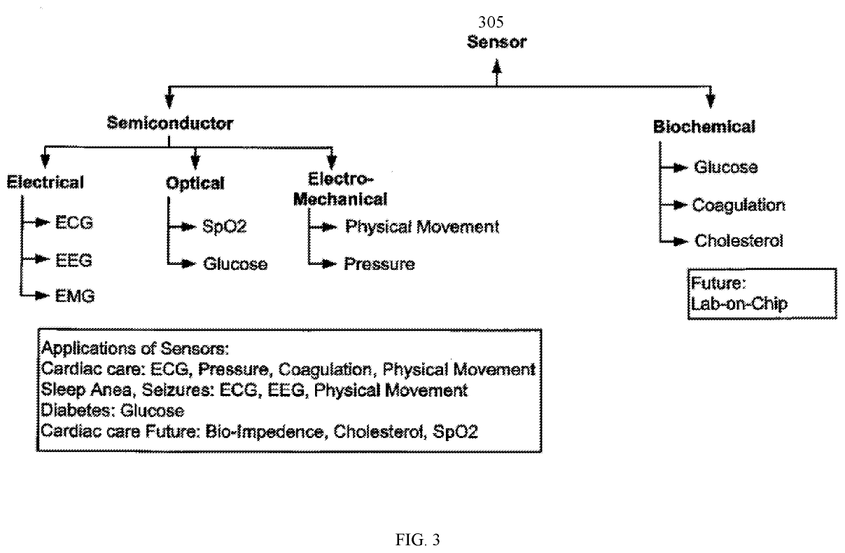

FIG. 3 illustrates an example of one or more sensors 305 for use with a biometric monitoring device (e.g., the biometric device 235 of FIG. 2) according to an example embodiment of the present disclosure. The sensors 305 can be a semiconductor or a biochemical type sensor. A semiconductor sensor can be an electrical, optical or elecro-mechanical sensor. In this way, a variety of systems can be designed for the management of diseases, health and fitness, by choosing the sensors that monitor the appropriate parameters associated with target applications.

Accordingly, the biometric monitoring device supports many classes of sensors for physiological data collection, such as:
Sensors (e.g., patches) contacting the patient's body through gels, etc.
Sensors embedded within the patient's body through surgical procedures.
Sensors probing the patient's body through micro-needle based skin punctures.
Sensors in close proximity of the patient's body (e.g., probing using a microwave or optical beam).
Sensors embedded in the therapeutic monitoring device 150 for periodic or occasional use.
Sensors that can read biochemical micro-fluidic test strips (e.g. glucose, blood coagulation rate) via electrical or optical sensor

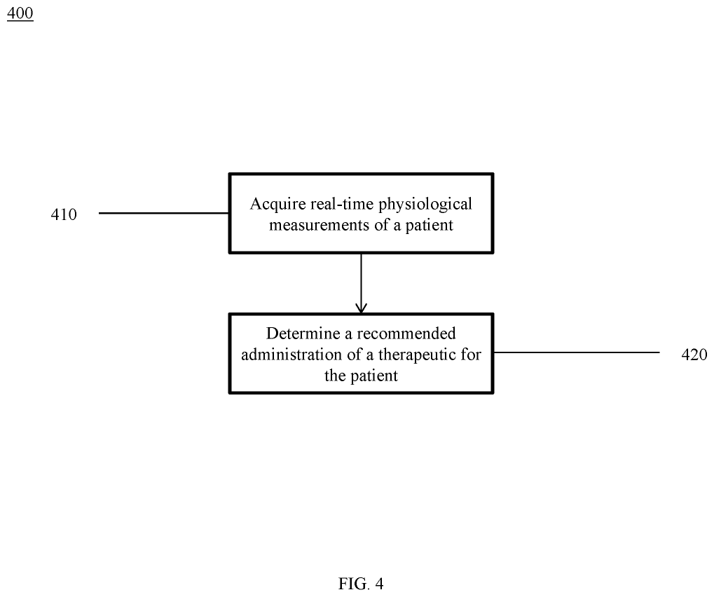

FIG. 4 is a flow diagram of a method 400 for physiological sensing and therapeutic administration according to an example embodiment of the present disclosure. The method 400, at 410, includes acquiring, via a physiological sensor (e.g., the sensor 205 of FIG. 2 and/or the sensor 305 of FIG. 3), real-time physiological measurements of a patient. At 420, the method 400 includes determining a recommended administration of a therapeutic for the patient based on the acquired real-time physiological measurements of the patient.

For example, a biometric monitoring device (e.g., the biometric monitoring device 235) may process (via the processing circuitry 215) the acquired “raw” sensor data. The biometric monitoring device then transmits the processed “raw” sensor data to a remote server (e.g., the remote server 120) to determine a recommended administration of a therapeutic for the patient. For instance, the remote server 120, via the PK server 130, analyzes the physiological sensor data in view of the patient's PK profile. In this way, the PK server 130 can quantify a rate at which the patient is metabolizing the therapeutic. Based on this rate, the PK server 130 can transmit a recommended administration of therapeutic to the therapeutic monitoring tool 150. The recommended administration of the therapeutic can include a timing and/or quantity component. For example, the timing and/or quantity component can be a function of the acquired real-time physiological measurements. The timing component can be a customized prophylactic dosing regimen of the therapeutic for the patient. In other aspects, the timing component can be an on-demand dosing of the therapeutic based on the time-varying therapeutic concentration level in the patient. In alterative aspects, the quantity component can be an amount of the therapeutic determined based on a function of the timing component and/or the acquired real-time physiological measurements.

Using the recommended administration of the therapeutic, the therapeutic monitoring tool 150 can control an administration of the therapeutic via the therapeutic administration device 140. In this way, the patient can be assured that therapeutic plasma concentration levels remain at safe levels. Advantageously, the patient can maintain a lifestyle that is not inhibited by a necessity of maintaining therapeutic plasma levels.

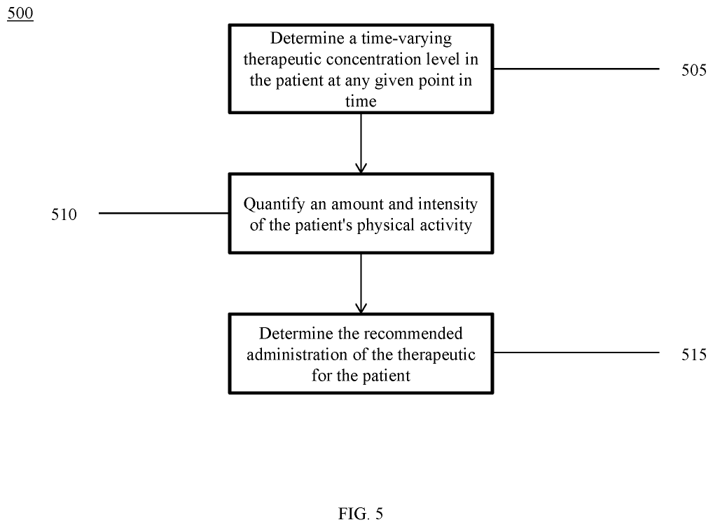

FIG. 5 is a flow diagram of a method 500 for determining a recommended administration of a therapeutic based on an amount and intensity of a patient's physical activity according to an example embodiment of the present disclosure. At 505, the method 500 includes determining, via a therapeutic monitoring tool (e.g., the tool 150 of FIG. 1), a time-varying therapeutic concentration level in the patient at any given point in time based on a PK profile of the patient and/or the acquired real-time physiological measurements of the patient. For instance, the therapeutic monitoring tool receives blood plasma measurements from a biochemical sensor (e.g., from the sensor(s) 305 of FIG. 3). The tool 150 then determines the therapeutic concentration level. Additionally, the tool 150 can receive hear rate and motion measurements from the electrical and electro-mechanical sensors (e.g., from the sensor(s) 305). Using this sensor data, a PK server (e.g., the PK server 130) can quantify an amount and intensity of the patient's physical activity.

The PK server, at 515, determines the recommended administration of the therapeutic for the patient. For instance, the PK server is able to use the current therapeutic plasma concentration level and the patient's activity level in view of the patient's PK profile to determine a rate at which the patient is and/or will metabolize the therapeutic.

The server can further determine a frequency of the activity and compare the frequency with historical activity measurements. Based on the comparison, the server can determine that the recommended an on-demand infusion (e.g., if the activity is random and infrequent) or an adjusted prophylactic infusion schedule (e.g., if the activity is frequent and a change from the patient's historical activity levels).

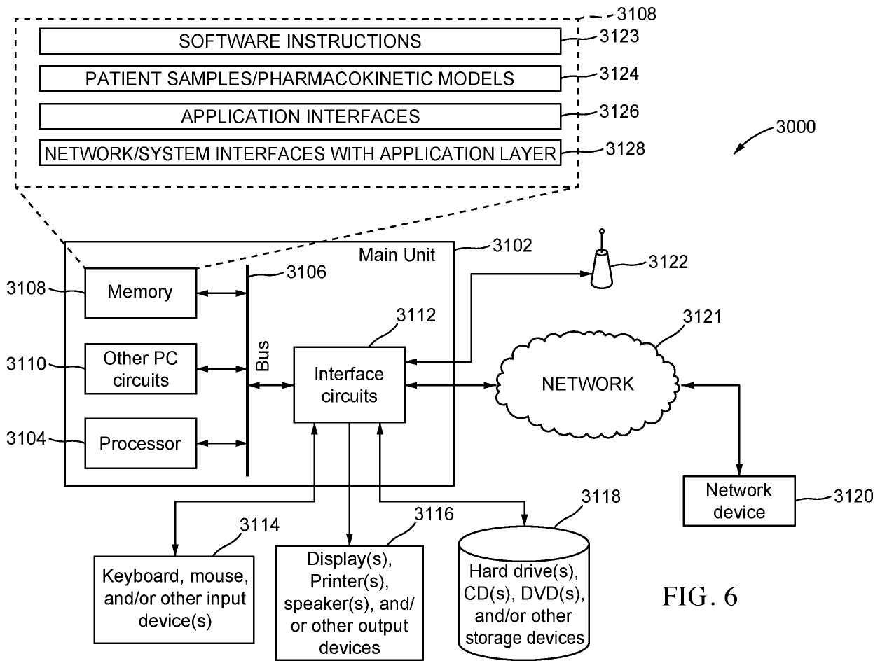

FIG. 6 is a detailed block diagram of an example computing device 3000. The computing device 300 can be any communication device such as a desktop computer, laptop computer, server system, cloud-based computing system, wireless transmit/receive unit (WTRU) (e.g., smartphone, tablet computer, mobile phone, personal digital assistant (PDA), etc.). Accordingly, the computing device 3000 can be, for example, the remote server 120, therapeutic monitoring tool 150, and/or the ecosystem monitoring system 180.

In this example, the device 3000 includes a main unit 3102. The main unit 3102 preferably includes one or more processors 3104 communicatively coupled by an address/data bus 3106 to one or more memory devices 3108, other computer circuitry 3110, and one or more interface circuits 3112. The processor 3104 may be any suitable processor, such as a microprocessor from the INTEL PENTIUM® or CORE™ family of microprocessors. The memory 3108 preferably includes volatile memory and non-volatile memory. Preferably, the memory 3108 stores a software program that interacts with the other devices in the environment 100, as described above. This program may be executed by the processor 3104 in any suitable manner. In an example embodiment, memory 3108 may be part of a “cloud” such that cloud computing may be utilized by the device 3000. The memory 3108 may also store digital data indicative of documents, files, programs, webpages, patient samples, pharmacokinetic models, patient pharmacokinetic profiles, etc. retrieved from (or loaded via) the device 3000.

The example memory devices 3108 store software instructions 3123, patient samples/pharmacokinetic models 3124, application interfaces 3126, user interface features, permissions, protocols, identification codes, content information, registration information, event information, and/or configurations. The memory devices 3108 also may store network or system interface features, permissions, protocols, configuration, and/or preference information 3128 for use by the device 3000. It will be appreciated that many other data fields and records may be stored in the memory device 3108 to facilitate implementation of the methods and apparatus disclosed herein. In addition, it will be appreciated that any type of suitable data structure (e.g., a flat file data structure, a relational database, a tree data structure, etc.) may be used to facilitate implementation of the methods and apparatus disclosed herein.

The interface circuit 3112 may be implemented using any suitable interface standard, such as an Ethernet interface and/or a Universal Serial Bus (USB) interface. One or more input devices 3114 may be connected to the interface circuit 3112 for entering data and commands into the main unit 3102. For example, the input device 3114 may be a keyboard, mouse, touch screen, track pad, track ball, isopoint, image sensor, character recognition, barcode scanner, microphone, and/or a speech or voice recognition system.

One or more displays, printers, speakers, and/or other output devices 3116 may also be connected to the main unit 3102 via the interface circuit 3112. The display may be a cathode ray tube (CRTs), a liquid crystal display (LCD), or any other type of display. The display generates visual displays generated during operation of the device 3000. For example, the display may provide a user interface and may display one or more webpages received from the device 3000. A user interface may include prompts for human input from a user of the device 3000 including links, buttons, tabs, checkboxes, thumbnails, text fields, drop down boxes, etc., and may provide various outputs in response to the user inputs, such as text, still images, videos, audio, and animations.

One or more storage devices 3118 may also be connected to the main unit 3102 via the interface circuit 3112. For example, a hard drive, CD drive, DVD drive, and/or other storage devices may be connected to the main unit 3102. The storage devices 3118 may store any type of data, such as identifiers, identification codes, registration information, patient samples, patient information, pharmacokinetic models, patient pharmacokinetic profiles, treatment regimes, statistical data, security data, etc., which may be used by the device 3000.

The computing device 3000 may also exchange data with other network devices 3120 via a connection to a network 3121 (e.g., the Internet) or a wireless transceiver 3122 connected to the network 3121. Network devices 3120 may include one or more servers, which may be used to store certain types of data, and particularly large volumes of data which may be stored in one or more data repository. A server may process or manage any kind of data including databases, programs, files, libraries, identifiers, identification codes, registration information, content information, patient samples, patient information, pharmacokinetic models, patient pharmacokinetic profiles, treatment regimes, statistical data, security data, etc. A server may store and operate various applications relating to receiving, transmitting, processing, and storing the large volumes of data. It should be appreciated that various configurations of one or more servers may be used to support, maintain, or implement the device 3000 of the environment 100. For example, servers may be operated by various different entities, including operators of the PK server 108, hospital systems, patients, drug manufacturers, service providers, etc. Also, certain data may be stored in the device 3000 which is also stored on a server, either temporarily or permanently, for example in memory 3108 or storage device 3118. The network connection may be any type of network connection, such as an Ethernet connection, digital subscriber line (DSL), telephone line, coaxial cable, wireless connection, etc.

Access to the device 3000 can be controlled by appropriate security software or security measures. An individual third-party client or consumer's access can be defined by the device 3000 and limited to certain data and/or actions. Accordingly, users of the environment 100 may be required to register with the computing device 3000.

While this disclosure has been particularly shown and described with references to example embodiments thereof, it will be understood by those skilled in the art that various changes in form and details may be made therein without departing from the scope of the present disclosure encompassed by the appended claims.

What is claimed is:

1. A physiological sensing and therapeutic administration system, the system comprising:
a sensor configured to acquire real-time physiological measurements of a patient; and
a pharmacokinetic (PK) server configured to determine a recommended administration of a therapeutic for the patient based on the acquired real-time physiological measurements of the patient, wherein the therapeutic has a time-varying concentration level in the patient.

2. The system of claim 1 further comprising:
a therapeutic monitoring tool configured to determine a time-varying therapeutic concentration level in the patient at any given point in time based on a PK profile of the patient and/or the acquired real-time physiological measurements of the patient.

3. The system of claim 2 wherein the recommended administration of the therapeutic includes a timing and/or quantity component, the timing and/or quantity component being a function of the acquired real-time physiological measurements.

4. The system of claim 3 wherein the timing component is a customized prophylactic dosing regimen of the therapeutic for the patient.

5. The system of claim 3 wherein the timing component is an on-demand dosing of the therapeutic based on the time-varying therapeutic concentration level in the patient.

6. The system of claim 3 wherein the quantity component is an amount of the therapeutic determined based on a function of the timing component and/or the acquired real-time physiological measurements.

7. The system of claim 3 wherein the PK server is further configured to quantify an amount and intensity of the patient's physical activity based on the physiological measurements.

8. The system of claim 7 wherein the PK server is further configured to determine the recommended administration of the therapeutic for the patient based on the amount and intensity of the patient's physical activity.

9. The system of claim 8 wherein the therapeutic dosing tool is configured to determine the time-varying therapeutic concentration level in the patient based on the amount and intensity of the patient's physical activity.

10. The system of claim 1 wherein the sensor is coupled to the patient's body at a location based on a type of the physiological measurements.

11. The system of claim 10 wherein the sensor includes circuitry and/or hardware that is specific to enable real-time acquisition of the type of the physiological measurements.

12. A physiological sensing and therapeutic administration method, the method comprising:
acquiring, via a physiological sensor, real-time physiological measurements of a patient; and
determining, via one or more processors, a recommended administration of a therapeutic for the patient based on the acquired real-time physiological measurements of the patient, wherein the therapeutic has a time-varying concentration level in the patient.

13. The method of claim 12 further comprising:
determining, via the one or more processors, a time-varying therapeutic concentration level in the patient at any given point in time based on a PK profile of the patient and/or the acquired real-time physiological measurements of the patient.

14. The method of claim 13 wherein the recommended administration of the therapeutic includes a timing and/or quantity component, the timing and/or quantity component being a function of the acquired real-time physiological measurements.

15. The method of claim 14 wherein the timing component is a customized prophylactic dosing regimen of the therapeutic for the patient.

16. The method of claim 14 wherein the timing component is an on-demand dosing of the therapeutic based on the time-varying therapeutic concentration level in the patient.

17. The method of claim 14 wherein the quantity component is an amount of the therapeutic determined based on a function of the timing component and/or the acquired real-time physiological measurements.

18. The method of claim 14 further comprising quantifying, via the one or more processors, an amount and intensity of the patient's physical activity based on the physiological measurements.

19. The method of claim 18 further comprising determining the recommended administration of the therapeutic for the patient based on the amount and intensity of the patient's physical activity.

20. The method of claim 19 further comprising determining, via the one or more processors, the time-varying therapeutic concentration level in the patient based on the amount and intensity of the patient's physical activity.

21. The method of claim 12 wherein the physiological sensor is coupled to the patient's body at a location based on a type of the physiological measurements.

22. The method of claim 12 wherein the physiological sensor includes circuitry and/or hardware that is specific to enable real-time acquisition of the type of the physiological measurements.
